(19) United States
(12) Patent Application Publication
(10) Pub. No.: US 2026/0263038 A1
(43) Pub. Date: Sep. 10, 2026

(54) IMAGING CATHETER AND MEDICAL SYSTEM

(71) Applicant: TERUMO KABUSHIKI KAISHA, Tokyo (JP)

(72) Inventor: Yasunori YAMASHITA, Fujinomiya Shizuoka (JP)

(21) Appl. No.: 19/671,168

(22) Filed: May 7, 2026

Related U.S. Application Data
(63) Continuation of application No. PCT/JP2024/039746, filed on Nov. 8, 2024.

(30) Foreign Application Priority Data
Nov. 10, 2023 (JP) 2023-192062

(51) Int. Cl. A61B 8/12; A61B 8/00

(52) U.S. Cl. CPC A61B 8/12; A61B 8/445; A61B 2562/0204

(57) ABSTRACT

An imaging catheter includes an inner tube including a first lumen, a sensor unit disposed at a distal end portion of the inner tube and including: a reinforcing body that includes a shaft portion, a distal end support portion disposed at a distal end of the shaft portion, and a proximal end support portion disposed at a proximal end of the shaft portion, a diameter of each of the distal and proximal end support portions being greater than an outer diameter of the shaft portion, and a film sensor including transducers, the film sensor surrounding the reinforcing body, an outer tube surrounding the inner tube at a position on a proximal side of the sensor unit and including a second lumen, and a bonding portion formed of a resin bonding agent and joining the sensor unit to the inner and outer tubes.

### CROSS-REFERENCE TO RELATED APPLICATION

This application is a continuation of International Patent Application No. PCT/JP2024/039746 filed Nov. 8, 2024, which is based upon and claims the benefit of priority from Japanese Patent Application No. 2023-192062 filed Nov. 10, 2023, the entire contents of which are incorporated herein by reference.

### TECHNICAL FIELD

The present disclosure relates to an imaging catheter and a medical system.

### BACKGROUND ART

In the treatment of a lesion such as a stenosis or an occlusion developed in a lumen organ such as a blood vessel, an imaging catheter using intravascular ultrasound (IVUS) is used as a medical device for acquiring a diagnostic image in order to observe the characteristics or the state after the treatment.

### SUMMARY

In a conventional imaging catheter, a distal end portion distal to a sensor unit for image acquisition is formed by connecting a fusion tube made of a thermoplastic resin or the like to a flange portion extending in a longitudinal direction on the distal side of the sensor unit and fusing the fusion tube by heat fusion or the like. Therefore, in the imaging catheter, a region (hard region) in which the fusion tube is melted and hardened is formed at the distal end portion by the length of the fusion tube. The distal end portion having the hard region has high rigidity and is not easily bent.

When inserted into a blood vessel that is a biological lumen, the imaging catheter advances to a target site along the shape of the blood vessel. However, since the imaging catheter has a long hard region at the distal end portion, the hard distal end portion may be caught on a blood vessel wall or the like when passing through a small-diameter blood vessel or the like, and there is room for improvement.

Embodiments of the present disclosure provide an imaging catheter and a medical system capable of improving the passability through a biological lumen by shortening the length in the longitudinal direction of a hard region of a distal end portion.

In one embodiment, an imaging catheter comprises: an inner tube including a first lumen through which a guidewire can pass; a sensor unit disposed at a distal end portion of the inner tube and including: a reinforcing body that includes a shaft portion, a distal end support portion disposed at a distal end of the shaft portion, and a proximal end support portion disposed at a proximal end of the shaft portion, a diameter of each of the distal and proximal end support portions being greater than an outer diameter of the shaft portion, and a film sensor on which a plurality of transducers are arranged, the film sensor surrounding the reinforcing body; an outer tube surrounding the inner tube at a position on a proximal side of the sensor unit and including a second lumen through which a signal line connected to the sensor unit can pass; and a bonding portion formed of a resin bonding agent and joining the sensor unit to the inner and outer tubes.

In the above-described imaging catheter, since the bonding portion is formed of the resin bonding agent without using a member such as a fusion tube, for example, the length in the longitudinal direction of the distal end bonding portion that joins the inner tube and the distal end support portion of the reinforcing body can be shortened. Therefore, the imaging catheter has improved passability through a biological lumen such as a blood vessel.

### BRIEF DESCRIPTION OF DRAWINGS

FIG. 1 is a schematic configuration diagram of a medical system including an imaging catheter according to an embodiment.

FIG. 2 is a view illustrating the imaging catheter.

FIG. 3 is a cross-sectional view of the imaging catheter.

FIG. 4A is a partial cross-sectional view of the periphery of a distal end portion of the imaging catheter.

FIG. 4B is a partial cross-sectional view of the periphery of a distal end portion of a conventional imaging catheter.

FIG. 5A is a view illustrating a first form of a fixing reinforcing portion of the imaging catheter shown in FIG. 1.

FIG. 5B is a view illustrating a second form of the fixing reinforcing portion of the imaging catheter shown in FIG. 1.

FIG. 5C is a view illustrating a third form of the fixing reinforcing portion of the imaging catheter shown in FIG. 1.

### DESCRIPTION OF EMBODIMENTS

Hereinafter, embodiments of the present disclosure will be described in detail with reference to the drawings. Embodiments described herein are shown to embody the technical idea of the present disclosure and do not limit the present disclosure. Other embodiments, examples, technical operations and the like that could be conceived by those skilled in the art without departing from the gist of the present disclosure are all included in the scope of the present disclosure and included in the invention recited in claims and the scope of equivalents thereof.

Furthermore, for the purpose of illustration and for ease of comprehension, the scale, aspect ratio, shape, and the like in the drawings attached may be changed from actual ones as appropriate and shown schematically. However, the drawings are merely illustrative and do not limit the interpretation of the present disclosure.

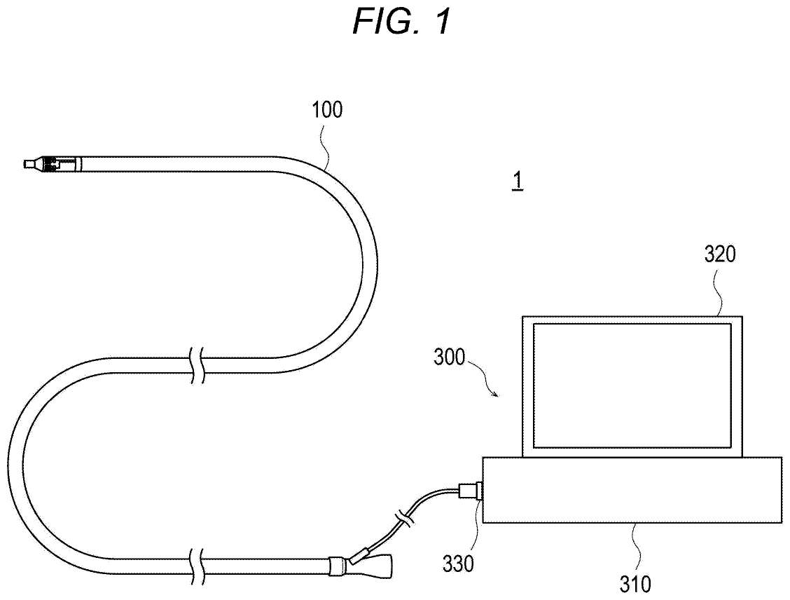

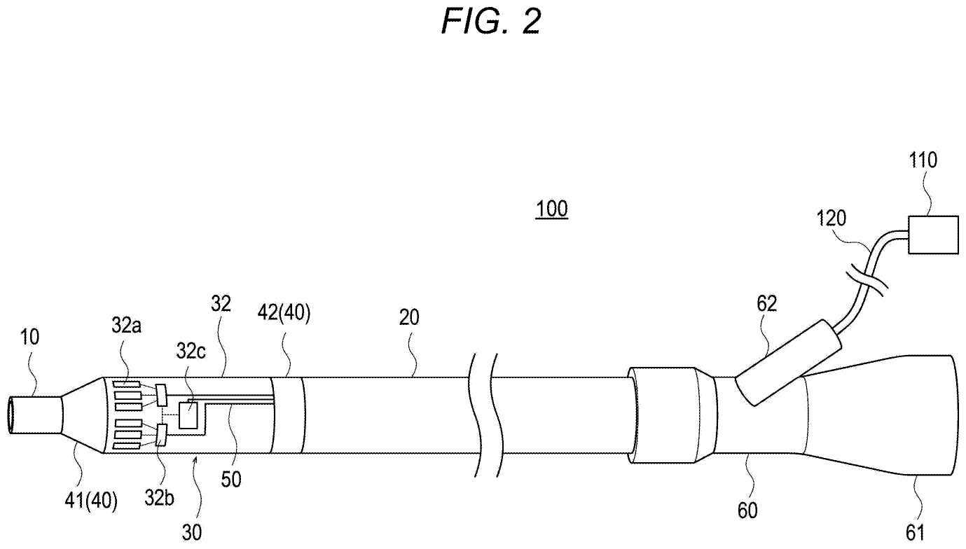

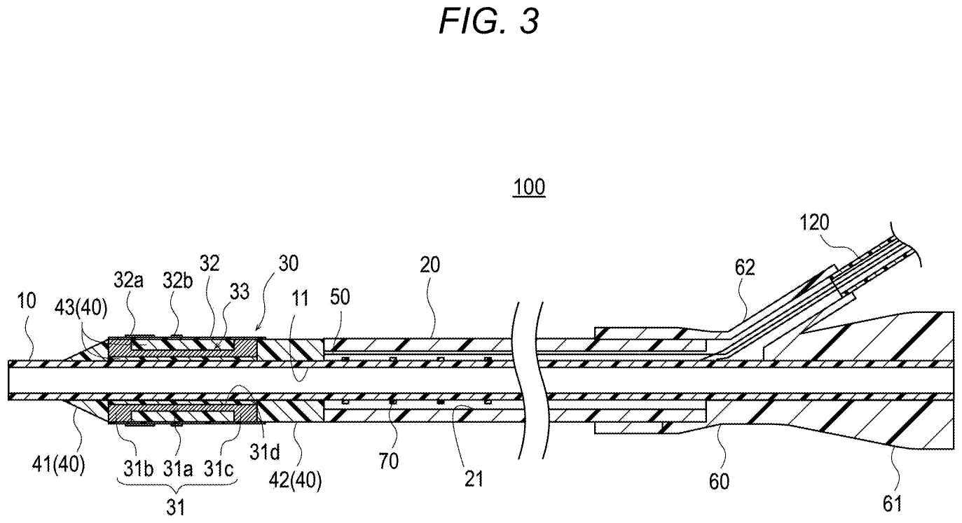

In the present specification, the following directions are defined for convenience of description. In FIG. 1, the “longitudinal direction” is a direction along the central axis of an imaging catheter 100. A “radial direction” is a direction away from or approaching the central axis in a cross-section (transverse cross-section) taken with the central axis of the imaging catheter 100 as a reference axis. A “circumferential direction” is a rotational direction with the central axis of the imaging catheter 100 as a reference axis.

The side of the imaging catheter 100 on which the hand hub portion 60 is disposed is referred to as a “proximal side”. In addition, a side of the imaging catheter 100 that is located on the opposite side to the proximal side and is introduced into the living body is referred to as a “distal side”. In addition, the “distal end portion” means a portion including a certain range from the distal end (most distal end) toward the proximal side, and the “proximal end portion” means a portion including a certain range from the proximal end (most proximal end) toward the distal side.

In one embodiment, the imaging catheter 100 is configured as an IVUS catheter utilizing intravascular ultrasound (IVUS).

In the present embodiment, a blood vessel is exemplified as a biological lumen to which the imaging catheter 100 is applied. However, the biological lumen to which the imaging catheter 100 is applied is not limited to only the blood vessel, and may be, for example, a biological organ such as a bile duct, a trachea, an esophagus, other digestive tracts, a urethra, or an ear, nose, and throat cavity.

As illustrated in FIG. 1, the medical system 1 includes the imaging catheter 100 and an external device 300. The medical system 1 is used when the imaging catheter 100 is inserted into a blood vessel of a patient, the sensor unit 30 is delivered to a lesion, an image (a tomographic image of the blood vessel or the like) captured at the lesion and around the lesion is output to an external device 300, and the property of the lesion is diagnosed.

### <Imaging Catheter>

The imaging catheter 100 includes an inner tube 10, an outer tube 20, a sensor unit 30, a bonding portion 40, a signal line 50, a hand hub portion 60, and a contrast unit 70. The imaging catheter 100 is configured as a so-called “over-the-wire catheter” in which a guidewire can be inserted and withdrawn from a catheter proximal end through the first lumen 11 of the inner tube 10. However, the imaging catheter 100 may be a so-called “rapid exchange type catheter”.

### <Inner Tube>

The inner tube 10 is an elongated tubular member having a first lumen 11 through which a guidewire extending from a distal end to a proximal end can be inserted. The inner tube 10 is configured such that a distal end thereof is located at a distal end of the imaging catheter 100, a proximal end thereof is located at a proximal end of the first port 61 of the hand hub portion 60, and a guidewire can be inserted and withdrawn.

A constituent material of the inner tube 10 is a material applicable to a catheter or the like, and for example, various thermoplastic elastomers such as a styrene-based thermoplastic elastomer, a polyolefin-based thermoplastic elastomer, a polyurethane-based thermoplastic elastomer, a polyester-based thermoplastic elastomer, a polyamide-based thermoplastic elastomer, a polyimide-based thermoplastic elastomer, a polybutadiene-based thermoplastic elastomer, a transpolyisoprene-based thermoplastic elastomer, a fluororubber thermoplastic elastomer, and a chlorinated polyethylene thermoplastic elastomer can be used.

### <Outer Tube>

The outer tube 20 is disposed so as to cover the inner tube 10 at a position on the proximal side of the sensor unit 30 so as to define the second lumen 21 through which the plurality of signal lines 50 can be inserted between the outer tube 20 and the inner tube 10. The proximal end of the outer tube 20 is located on the distal side of the distal end opening of the second port 62 inside the hand hub portion 60. As a result, the signal line 50 can be led out from the second port 62 after being inserted into the second lumen 21 from the sensor unit 30. The outer tube 20 is disposed coaxially with the inner tube 10.

The outer tube 20 can be made of, for example, the same material as the inner tube 10 exemplified above.

### <Sensor Unit>

The sensor unit 30 is disposed at the distal end portion of the inner tube 10 on the distal side of the outer tube 20. The sensor unit 30 includes a reinforcing body 31 and a film sensor 32. The sensor unit 30 transmits and receives ultrasonic waves in the biological lumen, and enables transmission of an electric signal to and from the external device 300 via the signal line 50 connected to each of the transducers 32a.

The reinforcing body 31 includes a cylindrical shaft portion 31a, a distal end support portion 31b disposed at a distal end of the shaft portion 31a and having a larger diameter than an outer diameter of the shaft portion 31a, and a proximal end support portion 31c disposed at a proximal end of the shaft portion 31a and having a larger diameter than the outer diameter of the shaft portion 31a. The reinforcing body 31 can be formed of a metal material. The reinforcing body 31 has a third lumen 31d into which the inner tube 10 can be inserted.

The film sensor 32 includes a flexible substrate in which electronic components are mounted on a film substrate, and an electronic circuit including a plurality of transducers 32a and the like is formed on an outer surface thereof. The film sensor 32 is wound around the distal end support portion 31b and the proximal end support portion 31c of the reinforcing body 31.

Each of the transducers 32a includes a piezoelectric element made of a piezoelectric material having piezoelectric characteristics such as ceramics or quartz capable of converting an electric signal into ultrasonic vibration. The transducers 32a constitute a phased array arranged in a ring shape along the circumferential direction of the reinforcing body 31 in a state where the film sensor 32 is wound around the reinforcing body 31. By using such a phased array type sensor, the sensor unit 30 can acquire a tomographic image of a blood vessel over a wide range in the circumferential direction at once without rotating the sensor unit 30. There are no particular limitations on the type, arrangement, and number of transducers 32a.

The plurality of transducers 32a have a function of transmitting ultrasonic waves based on an electric signal (pulse signal) into a body cavity and receiving ultrasonic waves reflected from a biological tissue in the body cavity. Each of the transducers 32a is disposed on the outer surface of the film sensor 32, and each is connected to the multiplexer 32b. The multiplexer 32b is a combinational circuit that selects one of a plurality of input lines and connects the selected input line to a single output line. Each of the transducers 32a is connected to each of the plurality of signal lines 50 via the multiplexer 32b. Each of the transducers 32a is configured such that each transmission/reception operation of transmission and reception can be sequentially switched by the multiplexer 32b. That is, the plurality of transducers 32a sequentially receive the respective reception signals and sequentially transmit the received signals via the multiplexer 32b. The multiplexer 32b is driven and controlled by a control IC32c formed on the film sensor 32.

### <Bonding Portion>

The bonding portion 40 joins the sensor unit 30 to the inner tube 10 and/or the outer tube 20. The bonding portion 40 includes a distal end bonding portion 41, a proximal end bonding portion 42, and a filling bonding portion 43. The distal end bonding portion 41, the proximal end bonding portion 42, and the filling bonding portion 43 are all formed of a resin bonding agent.

The distal end bonding portion 41 is disposed on the distal side of the sensor unit 30 so as to be adjacent to the distal end support portion 31b of the sensor unit 30 and to be in contact with the outer peripheral surface of the inner tube 10. Thus, the sensor unit 30 can be firmly bonded to the inner tube 10.

The distal end bonding portion 41 has a tapered surface 41a, the outer diameter of which decreases from the distal end support portion 31b of the sensor unit 30 toward the outer peripheral surface of the inner tube 10. Since the distal end bonding portion 41 has the tapered surface 41a, it is possible to improve the insertability of the imaging catheter 100.

The proximal end bonding portion 42 is disposed between the proximal end support portion 31c of the sensor unit 30 and the distal end portion of the outer tube 20 adjacent thereto. Thus, the sensor unit 30 can be firmly bonded to the outer tube 20.

The internal space 33 formed between the shaft portion 31a of the reinforcing body 31 and the wound film sensor 32 is filled with the filling bonding portion 43. Thus, the film sensor 32 can be firmly bonded to the reinforcing body 31. The third lumen 31d can also be filled with the filling bonding portion 43. Thus, the sensor unit 30 can be firmly bonded to the inner tube 10. In addition, the third lumen 31d is filled with the filling bonding portion 43 so as to connect the distal end bonding portion 41 and the proximal end bonding portion 42, whereby the bonding strength of the entire bonding portion 40 can be increased.

The resin bonding agent used as the bonding portion 40 has fluidity at the time of application, and has a property of being cured by a chemical change based on a curing treatment after application. As the resin bonding agent, for example, a UV curable adhesive or a thermosetting adhesive (epoxy adhesive or the like) can be suitably used.

The resin bonding agent constituting the distal end bonding portion 41 partially enters the interface between the reinforcing body 31 and the film sensor 32 on the distal side of the sensor unit 30. The resin bonding agent constituting the proximal end bonding portion 42 partially enters the interface between the reinforcing body 31 and the film sensor 32 on the proximal side of the sensor unit 30. As a result, the imaging catheter 100 can be firmly bonded only with the resin bonding agent without using a fusion tube or the like at the time of bonding the sensor unit 30 to the inner tube 10 and the outer tube 20, and the manufacturing process can be simplified. The distal end bonding portion 41, the proximal end bonding portion 42, and the filling bonding portion 43 constituting the bonding portion 40 may be formed of the same resin bonding agent, or may be formed of a plurality of resin bonding agents.

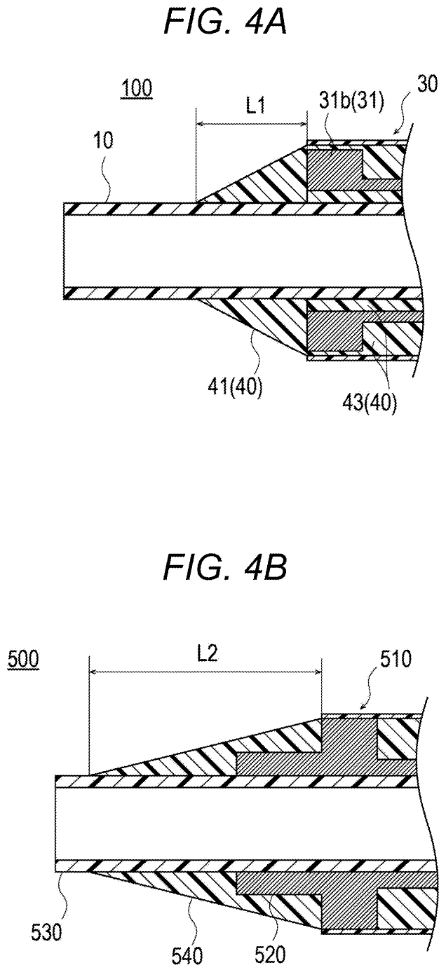

In the imaging catheter 100, the distal end bonding portion 41 can be formed only of a resin bonding agent as illustrated in FIG. 4A. The distal end bonding portion 41 forms a tapered surface 41a, and is formed such that the distal end support portion 31b and the outer peripheral surface of the inner tube 10 are joined. On the other hand, the configuration of the distal end portion of a conventional imaging catheter 500 is different from the configuration of the distal end bonding portion 41. FIG. 4B illustrates a configuration of the periphery of a distal end portion of the conventional imaging catheter 500 as a comparison. As illustrated in FIG. 4B, the distal end portion 501 of the imaging catheter 500 is provided with a flange portion 520 extending to the distal side of the sensor unit 510, and the fusion tube 540 is attached to the flange portion 520. Then, the imaging catheter 500 is configured such that the fusion tube 540 is thermally fused to bond the distal end portion of the sensor unit 510 and the outer peripheral surface of the inner tube 530. As a result, in the imaging catheter 500, a bonding portion 550 in which the distal end portion of the sensor unit 510 and the inner tube 530 are joined by the fusion tube 540 is formed.

As illustrated in FIG. 4A, the distal end bonding portion 41 of the imaging catheter 100 has a length L1 in the longitudinal direction. The length L1 in the longitudinal direction of the distal end bonding portion 41 corresponds to the length in the longitudinal direction of the hard region A1 of the distal end portion of the imaging catheter 100 that can be formed by the distal end bonding portion 41. On the other hand, as illustrated in FIG. 4B, the bonding portion 550 of the conventional imaging catheter 500 has a length L2 in the longitudinal direction. The length L2 of the bonding portion 550 in the longitudinal direction corresponds to the length of the hard region A2 in the longitudinal direction of the distal end portion of the imaging catheter 500 that can be formed by the bonding portion 550.

As illustrated in FIGS. 4A and 4B, the length L1 of the distal end bonding portion 41 in the longitudinal direction is shorter than the length L2 of the bonding portion 550 in the longitudinal direction (L1<L2). The distal end bonding portion 41 is formed of only a resin bonding agent, whereas the bonding portion 550 is formed of the fusion tube 540. Since the distal end bonding portion 41 is formed of a resin bonding agent, the length in the longitudinal direction can be adjusted to be short. On the other hand, since the fusion tube 540 is disposed by being connected to the flange portion 520, the bonding portion 550 inevitably becomes longer in the longitudinal direction by the length in the longitudinal direction of the fusion tube 540.

As described above, since the imaging catheter 100 has the distal end bonding portion 41 formed of the resin bonding agent, the length in the longitudinal direction is shorter than that of the bonding portion 550 formed of the fusion tube 540 of the conventional imaging catheter 500 (L1<L2). In the imaging catheter 100, the shorter the length L1 in the longitudinal direction of the distal end bonding portion 41, the shorter the length in the longitudinal direction of the hard region A1 of the distal end portion, and the blood vessel passability is improved. Therefore, the imaging catheter 100 can improve the operability in the blood vessel and suppress the blood vessel damage due to the contact with the inner wall of the blood vessel at the time of the advancing and retracting operation.

The length L1 of the distal end bonding portion 41 in the longitudinal direction is 0.1 mm or more and 5 mm or less, and preferably 0.1 mm or more and 2 mm or less from the viewpoint of blood vessel passability.

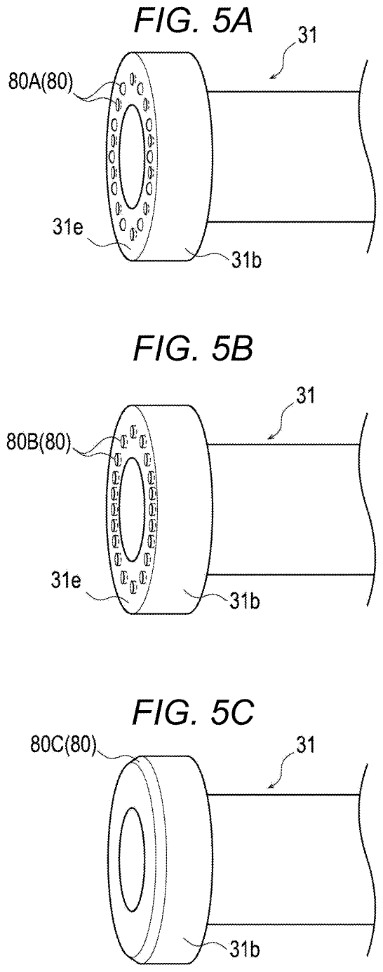

As illustrated in FIGS. 5A to 5C, the imaging catheter 100 can be configured to include a fixing reinforcing portion 80 that increases the fixing strength of the distal end bonding portion 41 with respect to the distal end support portion 31b of the reinforcing body 31. As in the first form illustrated in FIG. 5A, the second form illustrated in FIG. 5B, and the third form illustrated in FIG. 5C, the fixing reinforcing portion 80 can be formed in the distal end support portion 31b of the reinforcing body 31. Note that the following forms can be appropriately combined.

### (First Form)

As illustrated in FIG. 5A, the fixing reinforcing portion 80A according to the first form is formed of an uneven portion provided on the distal end surface 31e of the distal end support portion 31b. The contact area of the fixing reinforcing portion 80A with the distal end bonding portion 41 can be increased by the uneven shape. Since the distal end bonding portion 41 includes the fixing reinforcing portion 80A, the fixing strength with respect to the distal end support portion 31b increases, and peeling or the like during operation can be prevented.

### (Second Form)

As illustrated in FIG. 5B, the fixing reinforcing portion 80B according to the second form is formed of one or more holes formed in the distal end surface 31e of the distal end support portion 31b. The contact area of the fixing reinforcing portion 80B with the distal end bonding portion 41 can be increased by the resin bonding agent entering the hole. Since the distal end bonding portion 41 includes the fixing reinforcing portion 80B, the fixing strength with respect to the distal end support portion 31b increases, and peeling or the like during operation can be prevented.

### (Third Form)

As shown in FIG. 5C, the fixing reinforcing portion 80C according to the third form is formed of a tapered stepped portion formed on the outer peripheral surface of the distal end outer edge of the distal end support portion 31b so as to reduce in diameter toward the inner tube 10. The contact area of the fixing reinforcing portion 80C with the distal end bonding portion 41 can be increased by the tapered shape. Since the distal end bonding portion 41 includes the fixing reinforcing portion 80C, the fixing strength with respect to the distal end support portion 31b is increased, and peeling or the like during operation can be prevented. In addition, since the fixing reinforcing portion 80C has a tapered shape toward the inner tube 10, the tapered surface 41a is easily formed when the distal end bonding portion 41 is formed with the resin bonding agent.

In the fixing reinforcing portion 80C of the third form, the tapered stepped portion has a length in the longitudinal direction of 0.1 mm or more and 2 mm or less. As a result, the distal end bonding portion 41 can be formed to have an appropriate tapered surface while shortening the length in the longitudinal direction.

### <Signal Line>

The signal line 50 includes an element wire portion 51 made of a metal material having conductivity and contrast property (radiopacity), and has one end connected to the sensor unit 30 and the other end connected to the connector 110. Each of the signal lines 50 is connected to the multiplexer 32b and the control IC32c by soldering or the like. When the connector 110 and the connector 330 of the external device 300 are connected to each other, the signal line 50 can transmit and receive an electric signal between the sensor unit 30 and the external device 300. The signal line 50 is connected to the connector 110 in a state in which the portions exposed from the second port 62 of the proximal side hand hub portion 60 are collectively formed into the cable 120.

The signal line 50 may be configured to include a sheathed cable in which the element wire portion 51 is sheathed with a fluorine-based resin or the like, and a coaxial cable in which an outer conductor and an inner conductor formed of the element wire portion 51 are arranged at constant concentric intervals, and there may be a plurality of each. For example, when a coaxial cable is used as a reception-only line, the signal line 50 can reduce a transmission loss of an electric signal (imaging data) acquired by the sensor unit 30. The signal line 50 may be configured as a flexible cable in which the plurality of element wire portions 51 are sandwiched between resin films or the like, or a flat cable in which the plurality of element wire portions 51 covered with an insulating film are arranged in parallel in a band shape.

### <Hand Hub Portion>

The hand hub portion 60 has a first port 61 and a second port 62, and is connected to proximal end portions of the inner tube 10 and the outer tube 20.

The hand hub portion 60 is connected such that a proximal end opening of the first port 61 communicates with the first lumen 11 of the inner tube 10. As a result, the hand hub portion 60 allows insertion and withdrawal of the guidewire with respect to the inner tube 10. In the hand hub portion 60, the proximal end opening of the second port 62 and the second lumen 21 of the outer tube 20 are connected to communicate with each other. As a result, the hand hub portion 60 can lead the proximal end portions of the plurality of signal lines 50 out of the second port 62.

The hand hub portion 60 may be formed of, for example, a hard resin or a metal material applicable in the medical field.

### <Contrast Unit>

The contrast unit 70 is a radiopaque portion disposed near the distal end portion of the inner tube 10. The contrast unit 70 may be formed of, for example, a known metal material having radiopacity (X-ray opacity).

The contrast unit 70 can be arranged at constant intervals along the longitudinal direction of the inner tube 10 so as to function as a marker for measuring the distance from the sensor unit 30 to the lesion in the blood vessel, the length of the lesion, and the like. The arrangement interval of the contrast units 70 can be set to, for example, 10 mm to 40 mm.

The contrast unit 70 can be attached to the outer peripheral surface of the inner tube 10 by swaging or the like of a radiopaque metal component formed in a ring shape. The contrast unit 70 is not limited to the outer peripheral surface of the inner tube 10, and may be disposed in a state of being partially or entirely embedded in the tube wall of the inner tube 10. Note that the number, shape, arrangement interval, and the like of the contrast units 70 can be appropriately set according to the specifications of the imaging catheter 100.

### <External Device>

The external device 300 includes a control unit 310, a display device 320, and a connector 330.

The external device 300 is electrically connected to the imaging catheter 100 by connecting the connector 110 of the imaging catheter 100 to the connector 330. As a result, the external device 300 can transmit and receive an electric signal to and from the sensor unit 30 of the imaging catheter 100.

The control unit 310 is mainly composed of a processor such as a central processing unit (CPU), a memory, and an input/output unit, and controls the entire medical system 1. The control unit 310 outputs a control signal for emitting ultrasonic waves to the sensor unit 30 of the imaging catheter 100, inputs a detection signal from the sensor unit 30, and performs predetermined signal processing on the basis of the detection signal to acquire image data (tomographic image).

The control unit 310 displays information (video) based on the acquired image data on the display device 320.

As described above, the imaging catheter 100 is a device for acquiring ultrasonic tomographic images for diagnosis in a biological lumen, and includes: an inner tube 10 including a first lumen 11 through which a guidewire can be inserted; a sensor unit 30 disposed at a distal end portion of the inner tube 10, in which a plurality of transducers 32a that transmit and receive ultrasound waves in the biological lumen are arranged in a ring shape in a circumferential direction; a plurality of signal lines 50 that are connected to each of the transducers 32a and enable transmission of an electrical signal between the plurality of transducers 32a and the external device 300; an outer tube 20 disposed so as to cover the inner tube 10 at a position on the proximal side of the sensor unit 30 so as to define a second lumen 21 through which the plurality of signal lines 50 can be inserted between the outer tube 20 and the inner tube 10; a hand hub portion 60 which is connected to a proximal end portion of the inner tube 10 and a proximal end portion of the outer tube 20 and includes a first port 61 through which a guidewire can be inserted and withdrawn and a second port 62 through which the plurality of signal lines 50 are led out; and a bonding portion 40 that bonds the sensor unit 30 with the inner tube 10 and the outer tube 20. The sensor unit 30 includes: a reinforcing body 31 including a shaft portion 31a extending in the longitudinal direction, a distal end support portion 31b disposed at a distal end of the shaft portion 31a and having a larger diameter than an outer diameter of the shaft portion 31a, and a proximal end support portion 31c disposed at a proximal end of the shaft portion 31a and having a larger diameter than the outer diameter of the shaft portion 31a; and a film sensor 32 in which the plurality of transducers 32a are arranged on an outer surface and which is wound around the reinforcing body 31. The bonding portion 40 is formed of a resin bonding agent.

As illustrated in FIG. 4B, since the sensor unit 510 is bonded using the fusion tube 540, the distal end portion of the conventional imaging catheter 500 inevitably becomes longer in the longitudinal direction by the length in the longitudinal direction of the fusion tube 540. On the other hand, as illustrated in FIG. 4A, in the imaging catheter 100, since the distal end bonding portion 41 that joins the inner tube 10 constituting the bonding portion 40 and the distal end support portion 31b of the reinforcing body 31 is formed of a resin bonding agent, the length in the longitudinal direction can be shortened as compared with the bonding portion 550 (L1<L2). Therefore, the imaging catheter 100 improves operability in the blood vessel (blood vessel passability), and can suppress blood vessel damage due to contact with the inner wall of the blood vessel at the time of advancing and retracting operation.

What is claimed is:

1. An imaging catheter comprising:
an inner tube including a first lumen through which a guidewire can pass;
a sensor unit disposed at a distal end portion of the inner tube and including:
a reinforcing body that includes a shaft portion, a distal end support portion disposed at a distal end of the shaft portion, and a proximal end support portion disposed at a proximal end of the shaft portion, a diameter of each of the distal and proximal end support portions being greater than an outer diameter of the shaft portion, and
a film sensor on which a plurality of transducers are arranged, the film sensor surrounding the reinforcing body;
an outer tube surrounding the inner tube at a position on a proximal side of the sensor unit and including a second lumen through which a signal line connected to the sensor unit can pass; and
a bonding portion formed of a resin bonding agent and joining the sensor unit to the inner and outer tubes.

2. The imaging catheter according to claim 1, wherein
the bonding portion includes:
a distal end bonding portion having a tapered surface, an outer diameter of which decreases from the distal end support portion toward an outer peripheral surface of the inner tube, the distal end bonding portion bonding the distal end support portion to the inner tube;
a filling bonding portion with which a space between the shaft portion and the film sensor is filled; and
a proximal end bonding portion that bonds the proximal end support portion to the outer tube.

3. The imaging catheter according to claim 1, wherein
the reinforcing body includes an uneven portion formed on a distal end surface of the distal end support portion.

4. The imaging catheter according to claim 1, wherein
the reinforcing body includes one or more holes formed in a distal end surface of the distal end support portion.

5. The imaging catheter according to claim 1, wherein
the reinforcing body includes a tapered stepped portion formed on an outer peripheral surface of a distal end outer edge of the distal end support portion.

6. The imaging catheter according to claim 1, wherein
the bonding portion includes a distal end bonding portion that bonds the distal end support portion to the inner tube, and
a length of the distal end bonding portion in a longitudinal direction is 0.1 mm or more and 5 mm or less.

7. The imaging catheter according to claim 1, wherein
the resin bonding agent is an epoxy adhesive.

8. The imaging catheter according to claim 1, wherein
the sensor unit is free of a flange portion extending in a longitudinal direction on a distal side of the sensor unit, and
the bonding portion directly joins the distal end support portion to an outer peripheral surface of the inner tube.

9. The imaging catheter according to claim 1, wherein
the bonding portion includes a distal end bonding portion that joins the distal end support portion to an outer peripheral surface of the inner tube, and
the distal end bonding portion is formed exclusively of the resin bonding agent.

10. The imaging catheter according to claim 9, wherein
the resin bonding agent constituting the distal end bonding portion partially enters an interface between the reinforcing body and the film sensor on a distal side of the sensor unit.

11. The imaging catheter according to claim 9, wherein
a length of the distal end bonding portion in a longitudinal direction is 0.1 mm or more and 2 mm or less.

12. A medical system comprising:
an imaging catheter including:
an inner tube including a first lumen through which a guidewire can pass;
a sensor unit disposed at a distal end portion of the inner tube and including:
a reinforcing body that includes a shaft portion, a distal end support portion disposed at a distal end of the shaft portion, and a proximal end support portion disposed at a proximal end of the shaft portion, a diameter of each of the distal and proximal end support portions being greater than an outer diameter of the shaft portion, and
a film sensor on which a plurality of transducers are arranged, the film sensor surrounding the reinforcing body;
an outer tube surrounding the inner tube at a position on a proximal side of the sensor unit and including a second lumen through which a signal line connected to the sensor unit can pass; and
a bonding portion formed of a resin bonding agent and joining the sensor unit to the inner and outer tubes; and
an external device connectable to the imaging catheter and configured to transmit a control signal to the sensor unit and to acquire image data based on a detection signal received from the sensor unit.

13. The medical system according to claim 12, wherein
the bonding portion includes:
a distal end bonding portion having a tapered surface, an outer diameter of which decreases from the distal end support portion toward an outer peripheral surface of the inner tube, the distal end bonding portion bonding the distal end support portion to the inner tube;
a filling bonding portion with which a space between the shaft portion and the film sensor is filled; and
a proximal end bonding portion that bonds the proximal end support portion to the outer tube.

14. The medical system according to claim 12, wherein
the reinforcing body includes an uneven portion formed on a distal end surface of the distal end support portion.

15. The medical system according to claim 12, wherein
the reinforcing body includes one or more holes formed in a distal end surface of the distal end support portion.

16. The medical system according to claim 12, wherein
the reinforcing body includes a tapered stepped portion formed on an outer peripheral surface of a distal end outer edge of the distal end support portion.

17. The medical system according to claim 12, wherein
the bonding portion includes a distal end bonding portion that bonds the distal end support portion to the inner tube, and
a length of the distal end bonding portion in a longitudinal direction is 0.1 mm or more and 5 mm or less.

18. The medical system according to claim 12, wherein
the resin bonding agent is an epoxy adhesive.

19. The medical system according to claim 12, wherein
the sensor unit is free of a flange portion extending in a longitudinal direction on a distal side of the sensor unit, and
the bonding portion directly joins the distal end support portion to an outer peripheral surface of the inner tube.

20. The medical system according to claim 12, wherein
the bonding portion includes a distal end bonding portion that joins the distal end support portion to an outer peripheral surface of the inner tube, and
the distal end bonding portion is formed exclusively of the resin bonding agent.
